(19) United States
(12) Patent Application Publication
(10) Pub. No.: US 2004/0116825 A1
(43) Pub. Date: Jun. 17, 2004

(54) Method for hearing screening of newborn by means of steady state response evoked with high click rate

(76) Inventor: Ekkehard Sturzebecher, Petershagen (DE)

Correspondence Address: Dykema Gossett, Suite 300 West, 1300 I Street NW, Washington, DC, 20005-3306, US

(21) Appl. No.: 10/479,744

(22) PCT Filed: Jun. 7, 2002
(86) PCT No.: PCT/EP02/06262

(30) Foreign Application Priority Data
Jun. 7, 2001 (EP) 01610060.4

(51) Int. Cl. A61B 5/00

(52) U.S. Cl. 600/559

(57) ABSTRACT

The invention relates to a method for hearing screening of newborns by means of click evoked steady-state-potentials (SSR), where a click repetition rate in the range 60/s to 200/s is used, and that for the objective statistical detection of the SSR a Q-Sample Test is applied in the frequency domain, which not only takes into account the phase angle of the fundamental frequency but also the phase angle of the relevant higher harmonics in the calculation of the test value.

The invention relates to the area of objective determination of hearing ability, i.e. independent of the patient's cooperation, by means of a special form of auditory evoked potentials, the click-evoked steady state response.

Approximately 2 of 1000 newborn have a hearing disorder, which must be treated with a hearing aid or a cochlea implant, in order to provide a possibility of the development of speech. In Germany and also in other industrialized countries a child hearing disorder in average is diagnosed at the age of 31 month. Application of a hearing aid is then to late for normal speech development, as the sensible phase for the development of speech almost is passed.

A remedy can be achieved by an universal newborn screening carried out immediately after the birth. For this purpose portable automatic operating screening devices are required, that signals a pass or fail of the hearing test and thereby does not require any audiological qualification in order to interpret the registered data. The time consumption for the screening test must be limited. As a rule only one ear is tested for time and cost reasons, as the test of the second ear with the use of the known screening equipment requires a further examination.

Hearing screening methods are known, which based on the otoacoustic emission (OAE) and further the transitory evoked otoacoustic emissions (TEOAE) (DE 4441127 A1, devices: Echo-Screen from the company Fischer-Zoth Diagnosesysteme; Echocheck from the company Otodynamics, UK) as well as the distortion-product otoacoustic emissions (DPOAE) (DE 19623871 A1, device: ERO Scan from the company Etymotic Research, USA; AUDX from the company Bio-Logic Systems Corp., USA. The use of the OAE has the advantage that for the preparation for the registration only a single probe needs to be placed in the outer ear canal of the newborn and that the registration of the OAE does not involve a large time consumption. Use of the OAE for the hearing screening nevertheless has two major disadvantages: first only the functionality of the outer hair cells of the inner ear can be tested using the OAE. Hearing losses caused by a damage in the auditory nerve and brainstem are not detected. Second the frequency of the false positive test results (no registration of OAE, although hearing ability is normal) is relatively high. This increases the requirement for additional tests of the suspected newborn and thereby the costs of the hearing screening. Furthermore a hearing screening is known, which is based on the auditory brainstem response, ABR. (Stürzebecher et al., U.S. Pat. No. 6,071,246). By the use of ABR the mentioned disadvantages of the OAE are eliminated, however the registration of the ABR is so far when compared with the OAE significantly more time consuming.

A special form of the ABR is the SSR. The SSR is a periodic response to a periodic applied acoustic stimulus. A use of the SSR for a hearing screening avoids, like the ABR, the mentioned disadvantages of the OAE. The Amplitude-Modulation Following Response (AMFR) is a SSR that is evoked through an amplitude modulated continues tone (carrier). The hearing ability is tested at the frequency of the carrier, the response has the frequency of the modulation signal. A hearing screening based on the AMFR was suggested by Stürzebecher et al. (Stürzebecher E., Cebulla M., Pschirrer U.: Efficient stimuli for recording of the amplitude-modulation following response (AMFR), Audiology, accepted for publication). The time consumption for registration of the AMFR is approximately the same as the click evoked ABR. The AMFR does however, contrary to the ABR, allow for simultaneous testing of both ears without additional time consumption and supplies furthermore an additional frequency specific information.

A further known SSR is the 40-Hz-potential (Maurizi M., Almadori G., Paludetti G., Ottaviani F., Rosignoli M., Luciano R., 40-Hz steady state responses in newborns and in children, Audiology 1990; 29: 322-328), which occurs when the awake patient is subjected to click sequence of about 40/s and the ABR and the following large amplitude middle-latency response components are overlapping the response to the following clicks in such a way that a SSR with a large amplitude arises. For the hearing test of newborns the 40-Hz-Potential is not applicable, as the dominant middle latent parts are not developed in newborns and besides also later are reduced significantly during sleep.

Click evoked ABR using conventional registration techniques are known up to a maximum stimulus repetition rate of about 100/s (Lasky R E., Rate and adaptation effects on the auditory evoked brainstem response in human newborns and adults. Hearing Research 1997; 111: 165-176). Furthermore two special methods are known, which work with a higher click frequency, whereby however no SSR is generated. One of these method is the so-called Maximum-Length Technique (Lasky R E, Perlman J., Hecox K., Maximum length sequence auditory evoked brainstem responses in human newborns and adults. J. Am. Acad. Audiology 1992; 3: 383-389), by which it is possible by use of a special stimulus sequence to determine the individual not overlapping ABR, also at very high click frequencies. The method is thereby not directed towards obtaining a steady-state potential. A use of this method for hearing screening is discussed by Leung 1998 (Leung S., Slaven A., Thornton A R D., Brickley G J., The use of high stimulus rate auditory brainstem responses in the estimation of hearing threshold. Hearing Research 1998; 123: 201-205). The authors estimates that the improvement of the SNR as well as the reduction of time consumption only is limited. The second known use of click rates over 100/s is the so-called Chained-Stimuli technique (Hamill T A., Hussung R A., Sammeth C A., rapid threshold estimation using the “chained-stimuli” technique for auditory brainstem response measurements. Ear and hearing 1991; 12: 229-234) which in the second publication known in this context (Finkenzeller P. Der schnelle Stufenreiz zur Schwellenbestimmung. Aktuelle phoniatrisch-pädaudiologische Aspekte 1994; 2: 17-19) is designated as step stimulus. The step stimulus applied in a hearing screening device (Beraphon from the company MAICO Diagnoctic GmbH) comprises a sequence of 6 clicks with a time interval of 5 ms between the clicks (repetition rate 200/s), whereby the stimulus level for the individual click rises from 10 dBnHL for the first click in steps of ΔL=10 dB up to 60 dBnHL for the last click. Between the 6. click in one sequence and the 1. click in the following sequence there is a pause of 45 ms, i.e. repetition rate of the step stimulus sequence is 14.3/s. Further embodiments of the step stimulus are known, e.g. the step of ΔL=0 dB. The response to this special stimulus pattern is well suited for visual interpretation. For an objective statistical detection of the response components at 40 dBnHL or 35 dBnHL, as requested at a hearing screening, the pattern of the step stimulus is disadvantageous. A Steady-State-Potential can not be generated by this stimulus form.

The objective detection of the known SSR (both the AMFR and the click-evoked 40-Hz-Potential) takes place in the frequency domain. For this purpose several statistical methods are suitable (Stapells D R., Makeig S., Galambos R., Auditory steady-state responses: Threshold prediction using phase coherence. Electroencephalography and Clinical Neurophysiology 1987; 67: 260-270; Valdes J L, Perez-Abalo M C, Martin V, Savio G, Sierra C, Rodriguez E, Lins O. Comparison of statistical indicators for the automatic detection of 80 Hz auditory steady state response (AMFR). Ear and Hearing 1997; 18: 420-429) which as One-Sample Tests evaluate only the phase or the phase and the amplitude in a single spectral line. For this purpose the recorded time signal is transformed into the frequency domain. In the frequency spectrum present after the transformation the spectral line corresponding to the click repetition rate and to the modulation frequency, respectively, are searched and tested. The disadvantage of this known procedure is the limitation in the statistical testing to the fundamental frequency. SSR are normally not only represented by the fundamental frequency corresponding to the click rate and the modulation frequency, respectively, but also by one or more higher harmonics, which contain a significant part of the total signal power. An objective detection procedure that is only based on the fundamental frequency cannot be optimal.

The objective of the invention is to develop a method based on SSR for newborns screening that reduces the above mentioned disadvantages of the known solutions regarding the data and the recording of these as well as the statistical evaluation of the data and thereby has shorter examination time becoming comparable with the OAE.

A solution according to the invention is defined in claim 1. The method further presents the advantage of being applicable in both the frequency domain and the time domain. Further embodiments are defined in the dependent claims.

The concept of the invention involves deviating from the normal procedure that a click repetition rate of between 60/s and 200/s is used, preferably a click repetition rate of 90/s or 100/s, in order to determine the Steady-State-Potential. At these click rates the SSR is evoked with a high SNR. Deviating from the normal procedure the objective detection of the SSR is carried out with a so-called Q-Sample Test operating in the frequency domain, which not only takes into account the fundamental frequency, but also the higher harmonics, for objective potential detection. The use of a Q-Sample-Test on ABR data in the frequency domain is as such known (Stürzebecher et al. U.S. Pat. No. 6,071,246), however not in manner as suggested here. At the known method a stimulus repetiton rate of 59/s is used (distance between two consecutive stimuli about 16.95 ms). A time epoch of 16 ms containing the ABR is recorded. Because of a possible stimulus artifact, the first 1.0 ms of each epoch are rejected. The remaining epoch is filled with zeros to a length of 1024 ms and transformed into the frequency domain. In this process the periodic character of the response is not taken into account. A frequency spectrum is obtained, wherein in the interesting frequency range all spectral lines contains both signal energy and noise. The spectral SNR is therefore limited. Taking into account the periodic character of the response, due to the periodic stimulus application, will contrary to this at the registration and at the transformation into the frequency domain lead to a distribution of the noise energy on all spectral lines whereas the signal energy only is concentrated in the spectral lines of the fundamental frequency and the higher harmonics. This leads to a significant improvement of the spectral SNR.

Use of the solution according to the invention has the following advantages:

The use of a click repetition rate in the range mentioned above leads to a SSR, which is not mentioned in the literature with a large Signal-Noise-Ratio, which is a prerequisite for an easy objective detection of the response. Taking into account the periodic character of the SSR at the data sampling and processing means that the SSR can be represented in the spectrum by the fundamental frequency and a number of higher harmonics.

The taking into account of the higher harmonics due to the use of a suitable statistic test means in addition to the normal evaluation of the fundamental frequency that almost the total evoked signal but only a relatively small amount of noise is subjected to the test. This causes a further improvement of the response detection.

The improvement of the objective response detection due to the use of a suitable high click repetition rate as well as taking into account the relevant higher harmonics leads to a significant reduction of the examination time in the hearing screening.

Contrary to the ABR derived with conventional registration technique the SSR according to the invention allows for a simultaneous hearing test of both ears without additional time consumption by a single channel registration, as it is required for the test of only one ear.

Further details, characteristics and advantages of the invention appear from the following description of two embodiments referring to the attached drawings. In these

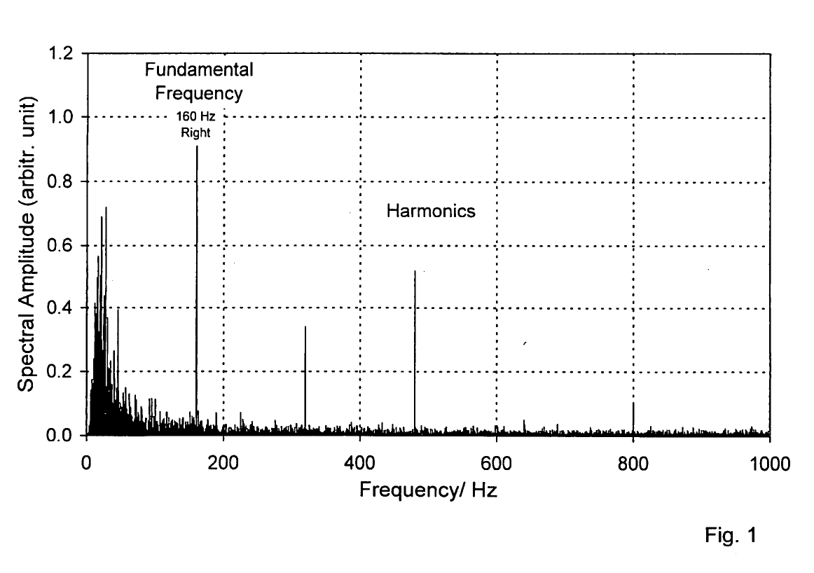

FIG. 1 shows the frequency spectrum of the SSR at acoustic click stimulation of the right ear;

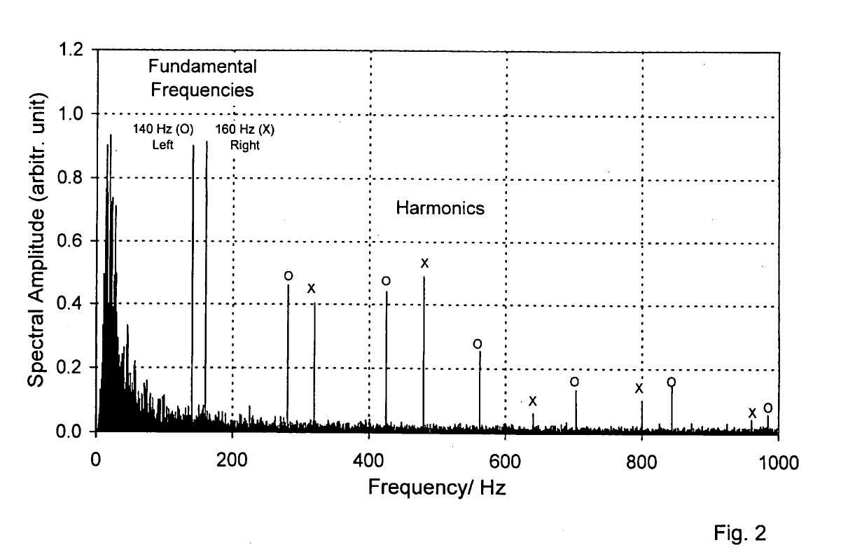

FIG. 2 shows the frequency spectrum of the two SSR at simultaneous acoustic click stimulation of both ears.

### EMBODIMENT 1

A newborn is tested, whether a normal hearing is present at the right ear. The examination takes place during the natural sleep after having been fed. The click repetition rate for the test of the right ear is 160/s. For the selected click rate a click sequence with a duration of 1 s is automatically calculated prior to the test and stored in a buffer memory in the signal processor of the screening device. The click sequence for the acoustic stimulation is created by cyclic read out of the buffer memory and after DA conversion supplied at a stimulus level of 40 dBnHL via an earphone with a tube connection (avoiding electrical stimulus artifacts) to the right ear. The DA conversion rate is 16384/s. During the acoustical stimulation the EEG is recorded through adhesive electrodes on the skin of the newborns head. The electrode placement is Vertex/Ipsilateral Mastoid, ground: forehead. The EEG is amplified and AD converted. The sampling frequency for the DA and the AD converter must be synchronized. In the present case the sampling frequency of the AD converter is 4096 Hz. The AD frequency is achieved by division (factor 4) of the DA frequency.

The digitized EEG is in the signal processor of the screening device continuously divided into parts (epochs) with a length of 1 s. A known device for artifact rejection makes sure that the epochs with artifacts are not used for the following evaluation. The artifact free epochs are transformed into the frequency domain using Fast Fourier Transformation (FFT). Phase angle and spectral amplitude of the spectral lines corresponding to the fundamental frequency and the related higher harmonics are stored in a data matrix. In order to cause in the frequency spectrum no side bands of the fundamental frequency and the higher harmonics, the epoch must only contain integer multiple of the SSR period. This is ensured through the selection of the epoch length and the click repetition rate. A FFT of the epochs is possible as the number of samples per epoch is a integer multiple of 2. FIG. 1 shows a frequency spectrum in which the fundamental frequency of the SSR (160 Hz) and the corresponding higher harmonics are marked. In order to achieve a high spectral SNR for the drawing, 200 epochs were averaged and the average was transformed by FFT. For the statistical testing, no averaging is necessary.

For the statistical test procedure the Q-Sample Uniform Scores Test (Mardia K V., Statistics of directional data, Academic Press London and New York 1972) is used. As soon as the first 10 epochs have been recorded and transformed, the first test is carried out. Simultaneous with the test the data sampling for further epochs is running. Each sampled epoch is transformed using the FFT. As soon as further 5 spectra have been added to the first 10 a new test is carried out. This sequential test procedure is continued until the SSR has been detected or until the maximum 200 epochs have been run. For the example it is assumed that the SSR of the right ear has been detected already with 25 epochs (after 25 s). After 25 seconds the screening device signals a “PASS”. The screening examination is hereby completed after 25 seconds. Assuming an intervention requiring hearing loss of the right ear the required test duration until the decision “FAIL” is 200 s.

### EMBODIMENT 2

A newborn is tested, whether a normal hearing is present at both ears. The examination takes place during the natural sleep after having been fed. The click repetition rate for the test of the right ear is 160 clicks/s, for the left ear 140/s. For each of the two click rates a click sequence with a duration of 1 s automatically calculated prior to the test and stored in a buffer memory in the signal processor of the screening device. The two click sequences for the acoustic stimulation are created by cyclic request to the buffer memory and after DA conversion with a stimulus level of 40 dBnHL via each a earphones with a tube connection (avoiding electrical stimulus artifacts) supplied to the right ear and to the left ear. The DA conversion rate is 16384/s.

During the acoustical stimulation the EEG is recorded through adhesive electrodes on the skin of the newborns head. The electrode placement is Vertex/neck, ground: forehead. The EEG is amplified and AD converted. The sampling frequency for the DA and the AD converter must be synchronized. In the present case the sampling frequency of the AD converter is 4096 Hz. The AD frequency is achieved by division (factor 4) of the DA frequency.

The digitized EEG is in the signal processor of the screening device continuously divided into parts (epochs) with a length of 1 s. A known device for artifact rejection makes sure that epochs with artifacts are not used for the following evaluation. The artifact free epochs are transformed into the frequency domain using Fast Fourier Transformation (FFT). Phase angle and spectral amplitude of the spectral lines corresponding to the fundamental frequency and the related higher harmonics are stored in two data matrices. In order to obtain in the frequency spectrum no side bands of the fundamental frequency and the higher harmonics, the epochs must only contain integer multiples of the periods of the SSR from the right and the left side. This is ensured through the selection of the epoch length and the click repetition rate. A FFT of the epochs is possible as the number of samples per epoch is an integer multiple of 2. FIG. 2 shows a frequency spectrum in which the fundamental frequency of the two SSR (140 Hz and 160 Hz) and the corresponding higher harmonics are marked. In order to achieve a high spectral SNR for the drawing, 200 epochs were averaged and the average was transformed by FFT. For the statistical testing, no averaging is necessary.

For the statistical test procedure the Q-Sample Uniform Scores Test (Mardia K V., Statistics of directional data, Academic Press London and New York 1972) is used. As soon as the first 10 epochs have been recorded and transformed, the first test is carried out, whereby the influence responses for the spectral lines of the right and the left ear are tested separately. Simultaneous with the test the data sampling for further epochs is running. Each sampled epoch is transformed using the FFT. As soon as further 5 spectra have been added to the first 10 a new test is carried out. This sequential test procedure is continued until both SSR (right and left) have been detected or until the maximum 200 epochs have been run. For the example it is assumed that the SSR of the rights ear has been detected already after 25 epochs (25 s) and of the left ear after 30 epochs (30 seconds). After 25 seconds the screening device signals a “PASS” for the right ear and after an additional 5 seconds also for the left ear a “PASS”. The screening examination is hereby completed after 30 seconds. At an intervention requiring hearing loss of one or both ears the required test duration until the signal “FAIL” is 200 s, whereby by a unilateral hearing loss a “PASS” has been signaled for the healthy ear earlier in the procedure.

1. Method for hearing screening of newborns by means of click evoked steady-state-potentials (SSR), characterized in that for the objective statistical detection of the SSR a Q-Sample Test is applied in the frequency domain or in the time domain, which not only takes into account the phase angle of the fundamental frequency but also the phase angle of the relevant higher harmonics in the calculation of the test value.

2. Method according to claim 1, characterized in that a click repetition rate in the range 60/s to 200/s is used.

3. Method according to claim 2 characterized in that a click repetition rate in the range of 60-160/s, preferably 90-100/s is used.

4. Method according to claim 1, 2 or 3, characterized in that for the objective statistical SSR detection a Q-Sample Test is applied in the frequency domain, which besides the phase angles of the fundamental frequency and the relevant higher harmonics also takes into account the spectral amplitude of the fundamental frequency and the relevant higher harmonics in the calculation of the test value.

5. Method according to claim 1, 2 or 3, characterized in that for the objective statistical SSR detection a One-Sample Test id applied in the frequency domain or in the time domain, which only takes into account the phase angle of the fundamental frequency in the calculation of the test value.

6. Method according to claim 1, 2 or 3, characterized in that for the objective statistical SSR detection a One-Sample Test is applied in the frequency domain or in the time domain, which besides the phase angle also takes into account the spectral amplitude of the fundamental frequency in the calculation of the test value.

7. Method according to any of the claims 1-6, characterized in that for a simultaneous hearing screening of both ears, two click sequences with different click repetition rates in the range between 60/s and 160/s are generated and that for the statistical detection of both responses in the same spectrum the statistical test is applied both to the spectral lines representing the SSR of the right ear and to the spectral lines representing the SSR of the left ear.
